(19) United States
(12) Patent Application Publication
(10) Pub. No.: US 2002/0132346 A1
(43) Pub. Date: Sep. 19, 2002

(54) Use of RNA interference for the creation of lineage specific ES and other undifferentiated cells and production of differentiated cells in vitro by co-culture

(76) Inventor: Jose Cibelli, Amherst, MA (US)

Correspondence Address: PILLSBURY WINTHROP, LLP, P.O. BOX 10500, MCLEAN, VA, 22102, US

(21) Appl. No.: 10/092,598

(22) Filed: Mar. 8, 2002

Related U.S. Application Data
(60) Provisional application No. 60/273,974, filed on Mar. 8, 2001.

(51) Int. Cl. C12N 5/08; C12N 15/87

(52) U.S. Cl. 435/455; 435/366

(57) ABSTRACT

Methods for making human ES cells and human differentiated cells and tissues for transplantation are described, whereby the cells and tissues are created following somatic cell nuclear transfer. The nuclear transfer donor is genetically modified prior to nuclear transfer such that cells of at least one developmental lineage are de-differentiated, i.e., unable to develop, thereby resolving the ethical dilemmas involved in reprogramming somatic cells back to the embryonic stage. The method concomitantly directs differentiation such that the desired cells and tissues may be more readily isolated.

### FIELD OF INVENTION

The present invention relates to methods of directing the differentiation of embryonic cells and embryonic stem (ES) cells along a particular lineage. The invention is also concerned with precluding the differentiation of embryonic cells and ES cells along particular lineages such that the embryonic cells and ES cells of the invention are incapable of developing into an embryo or fetus. Such embryonic and ES cells are especially useful in the field of human therapeutic cloning, for isolating desired differentiated cells and tissues for transplantation and other therapies while at the same time avoiding the ethical dilemmas associated with human cloning.

### BACKGROUND OF THE INVENTION

The past decade has seen many significant developments in the fields of nuclear transfer technology and embryonic development. Successes in the cloning field range from the introduction of Dolly the sheep in 1997 to the cross-species cloning of a guar using an adult differentiated donor cell and an enucleated bovine oocyte in 2000 (see Lanza et al., November 2000, “Cloning Noah's Ark,” Scientific American). Advances were made as well in the area of human embryonic research as two separate groups reported recently the isolation of human embryonic stem cells capable of differentiating into all the different cells of the body (see Shamblott et al., Nov. 10, 1998, “Derivation of pluripotent stem cells from cultured human primordial germ cells,” Proc. Natl. Acad. Sci. USA 95(23):13726-31; see also Thomson et al., Nov. 6, 1998, “Embryonic stem cell lines derived from human blastocysts,” Science 282(5391): 1145-47). As scientists begin to unravel the molecular processes involved in nuclear reprogramming and embryonic development, the potential for using the technology as a means to effectuate therapeutic cloning of autologous transplantation tissues for humans draws tantalizing close.

The impact that human ES cells and somatic cell nuclear transfer will have on transplantation medicine is unprecedented. Because of their capacity for unlimited growth in culture, human ES cells have the potential to provide an unlimited source of any cell in the body. Such cells could then be used to replace or supplement cells in a patient in need of such treatment, for instance a cancer patient needing a transfusion of blood cells following radioimmunotherapy or chemotherapy. Such differentiated cells could also be used to engineer new tissues, for instance for patients in need of liver or heart transplants or cardiac patches. Human ES cells derived from somatic cell nuclear transfer provide even further advantages, because such cells have the same genetic makeup as the patient. Therefore, there is no need to protect against transplant rejection of differentiated cells derived from cloned human ES cells using immunosuppressive treatments, which weaken the patient's immune system and cause the potential for further medical problems. Moreover, the donor cells for somatic cell nuclear transfer can be readily carried in culture, thereby facilitating genetic modification such as deletion of disease-related genes or addition of therapeutic genes prior to nuclear transfer.

The development of human ES cells will also revolutionize pharmaceutical research and development when unlimited sources of normal human differentiated cells become available for drug screening and testing, drug toxicology studies and new drug target identification. Cellular models of human disease will be more readily developed, and will provide advantages over the immortalized cell lines that are currently available, which are capable of long term growth only because of changes in genetic structure that could potentially affect the interpretation of data gleaned from such cells. ES cells will also serve as valuable resources for the study of human embryonic development, and will help researchers understand and treat fertility disorders, prevent premature births and miscarriages, and diagnose and prevent birth defects (see “The First Derivation . . . ,” supra).

Despite the promise that human ES cells and cloned therapeutic tissues hold for the understanding of human development and the creation of tissues for transplantation, the ethical debate over human cloning has been growing fervently as the pace of technology progresses. Some of the ethical arguments are fueled by “irrational fantasies and fears, based mainly on the misconception that genetic identity means identical twin personalities” (M. Revel, 2000, “Ongoing research on mammalian cloning and embryo stem cell technologies: bioethics of their potential medical applications, lsr. Med. Assoc. J. 2 Suppl: 8-14). Other arguments stress that the isolation of specific cells and tissues from nuclear transfer-derived embryos and human embryonic stem cells each involves the destruction of a potential human life and are therefore objectionable on moral grounds (see E. Young, February 2000, “A time for restraint,” Science 287 (5457): 1424; see also Coghlan and Boyce, “Put it to the vote,” New Scientist, Aug. 19, 2000). Such arguments have contributed to the current constraints on available funding for therapeutic cloning research, and perpetuate the public's misconception and aversion to therapeutic cloning despite the fact that the goal is to direct the development of particular tissues using ES cells rather than form an entire embryo (see National Institutes of Health Guidelines for Research Using Human Pluripotent Stem Cells,” 65 FR 51976, Aug. 25, 2000).

The fact remains, however, that an embryo having the potential to develop into a human being is destroyed using the techniques that are currently available for making human ES cells. For instance, one group that recently reported the isolation of human embryonic stem (ES) cells isolated the ES cells from the gonadal ridge and mesenteries of a donated 5-9 week human embryo resulting from a terminated pregnancy (Gearhart, supra). The other group derived their ES cells from in vitro fertilized blastocysts which were donated after informed consent (Thomson, supra). Although researchers predict that it will one day be possible to “reprogram” a patient's cells with chemicals and convert them directly into tissue for transplantation thereby sidestepping the formation of a short-lived embryo, some have stressed that the only way that the necessary chemical signals can be deciphered is by experimenting on stem cells from human embryos (see Coghlan, “Back to the Source,” New Scientist, Aug. 19, 2000). Thus, it would be quite valuable with regard to funding as well as for promoting public support and education if the necessary experimentation using human embryonic cells and ES cells could be performed using cells that have no potential for human life.

Other groups have proposed various solutions for addressing this ethical dilemma. For instance, researchers at Geron BioMed, a company launched by the team that cloned Dolly at the Roslin Institute near Edinburgh, believes that the use of human ES cells will help address the ethical dilemma because such cells cannot develop into an embryo (see Coghlan, “Cloning with out embryos: An ethical obstacle to cloned human tissue may be about to disappear,” New Scientist, Jan. 29, 2000). Indeed, the current techniques for isolating ES cells involve removal of cells from the inner cell mass of a blastocyst, whereas the trophoblast cells required for implantation in the uterus are left behind. Nevertheless, ardent pro-life groups might still object to the use of such ES cells because they are derived from a human embryo in the first place. Moreover, the only way to develop cells and tissues for transplantation that have an identical or similar genetic make-up as the patient in need of transplant would be to use the patient's own cells to effect somatic cell nuclear transfer, thereby isolating ES cells from a newly derived blastocyst, or use embryos made by in vitro fertilization (IVF) that have a partial genotype match.

Geron has also suggested, however, that ES cells could be used as nuclear transfer recipients in lieu of eggs. Therefore, the idea is to use enucleated ES cells rather than oocytes to derive ES cells having the same genetic makeup as a transplant recipient, thereby forming ES cells specific for the patient without generating a short-lived embryo. In fact, Geron's proposed approach was inspired by a report by Azim Surani and colleagues at the Wellcome /CRC Institute of Cancer Research and Developmental Biology at Cambridge, who reported in 1997 the reprogramming of mouse thymocytes after fusing them with mouse embryonic germ cells. Surani has cautioned, however, that gutted stem cells may not make all the necessary factors for reprogramming like oocytes do (see Coghlin, Jan. 29, 2000, supra). Furthermore, such techniques would still require the use of ES cells that were initially derived from a human embryo.

Others have argued that research on human pluripotent ES cells is unnecessary because stem cells from adults, umbilical cords and placentas could be used instead (see NIH Guidelines, supra). However, adult stem cells may have a more limited potential than embryonic stem cells. For instance, adult stem cells that give rise to some cell lineages in the body have not yet been identified, i.e., cardiac stem cells and pancreatic islet stem cells, therefore, some cell types cannot yet be isolated via differentiation of adult stem cells (see NIH Guidleines, supra). Furthermore, adult stem cells are present only in minute quantities, are difficult to isolate and purify, and their numbers may decrease with age. They are also more difficult to maintain in culture with losing their undifferentiated state. Any genetic defect that contributed to the patient's disorder would likely also be present in the patient's stem cells as well. In fact, adult stem cells are likely to contain more DNA abnormalities caused by exposure to sunlight, toxins and errors in DNA replication than are embryonic stem cells whereas ES cells maintain a structurally normal set of chromosomes even after prolonged growth in culture (see “The First Derivation . . . ,” supra). Adult stem cells may also have a more limited life span than ES cells, particularly cells generated from nuclear transfer derived embryos where the telomeres have been shown to be increased in length in comparison to non-cloned controls in mammalian studies. U.S. application Ser. No. 09/527,026 filed on Mar. 16, 2000 and 09/520,879 filed on Apr. 5, 2000 and 09/856,173 filed on Sep. 6, 2000 describe the results and implications of this phenomenon, and are hereby incorporated by reference in its entirety. In contrast, other stem cells express telomerase at low levels or only periodically and therefore age and stop dividing with time (“The First Derivation . . . ,” supra).

U.S. Pat. Nos. 5,753,506 and 6,040,180 (assigned to CNS Technology, Inc.) describe the directed differentiation of and the in vitro generation of differentiated neurons from embryonic and multipotent CNS stem cells. The methods reportedly allowed for the directed differentiation of neural cells in vitro using specific culture conditions, however, the only means disclosed for deterring embryonic development is to separate the desired precursor cells away from the other lineages. Such a technique in the context of ES cell differentiation would not address the ethical dilemmas raised by the using human ES cells in the first place.

There are further examples of in vitro differentiation of multipotent and pluripotent stem cells in the literature. ES cells derived from blastocyst and post-implantation embryos have also been allowed to differentiate into cultures containing either neurons or skeletal muscle (Dinsmore et al., “High Efficiency Differentiation of Mouse Embryonic Stem Cells into Either Neurons or Skeletal Muscle in vitro” Keystone Symposium (Abstract H111) J. Cell. Biochem. Supplement 18A:177 (1994)), or hematopoietic progenitors (Keller et al., “Hematopoietic Commitment During Embryonic Stem Cell Differentiation in Culture” Mol. Cell. Biol. 13:473-486 (1993); Biesecker and Emerson, “Interleukin-6 is a Component of Human Umbilical Cord Serum and Stimulates Hematopoiesis in Embryonic Stem Cells in vitro” Exp. Hematology 21:774-778 (1993); Snodgrass et al., “Embryonic Stem Cells and in vitro Hematopoiesis” J. Cell. Biochem. 49:225-230 (1992); and Schmitt et al., “Hematopoietic Development of Embryonic Stem Cells in vitro: Cytokine and Receptor Gene Expression” Genes and Develop. 5:728-740 (1991)). However, in none of these examples is the differentiation of the pluripotent stem cell genetically directed down a particular pathway or deterred from a particular pathway. Instead, they are allowed to differentiate randomly into a mixed population of terminally differentiated cells. Thus, there is no means of isolating a substantially pure population of progenitor cells of a desired cell lineage, and again the ethical dilemmas are not resolved.

U.S. Pat. No. 5,639,618 (assigned to Plurion, Inc.) discloses methods for isolating lineage specific stem cells in vitro, wherein a pluripotent embryonic stem cell is transfected with a DNA construct comprising a regulatory region of a lineage specific gene operably linked to a DNA encoding a reporter protein, and the transfected pluripotent embryonic stem cell is cultured under conditions such that the pluripotent embryonic stem cell differentiates into a lineage specific stem cell. However, the proposed methods result only in the molecular “tagging” of cells of the desired lineage, which cells must then be separated from other cells in the culture by virtue of the reporter protein. Thus, although the methods permit the identification of specific cell lineages derived from embryonic stem cells, the development of unwanted or unnecessary lineages is not deterred in such a way that an embryonic cell having no potential for life is employed. In fact, the ES cells used to construct the cell lines in this patent were derived from primordial germ cells isolated from post-implantation embryos. Hence, the methods do not address the ethical dilemmas associated with using human ES cells for generating transplantation cells and tissues.

U.S. Pat. No. 5,863,774 (assigned to The General Hospital Corporation and President and Fellows of Harvard College) reports a method for ablating certain cell types in Drosophila fertilized embryos using ribozymes expressed from cell-specific promoters. Although the use of the cell ablation technique was disclosed as being applicable to the study of Drosophila embryogenesis, sex selection in plants and protection of mammals and plants against viruses, no mention was made of using the disclosed cell ablation techniques in the context of human therapeutic cloning or somatic cell nuclear transfer.

Thus, it is clear that human embryonic stem cells provide advantages over other stem cells with regard to generating tissue for transplantation and other differentiated cells. It is also clear that the use of such cells in the context of somatic cell nuclear transfer has the potential to provide tissue compatible transplant material, because such ES cells can be derived using the patient's own genetic material. However, it is also clear that ethical and moral concerns regarding this technology continue to be problematic despite the significant advantages to be gained. It would be desirable to develop human ES cells using nuclear transfer that do not give rise to ethical or moral concerns. It would also be desirable to direct such cells to develop into particular cell lineages, while at the same time precluding the use of cells having any potential for human life.

### SUMMARY OF INVENTION

The present invention fills in the holes present in the prior art by providing a means for studying and directing the differentiation of embryonic cells and ES cells without ever having a short-lived embryo as an intermediary. Thus, the methods of the invention should resolve the ethical dilemmas associated with human somatic cell nuclear transfer as a means to generate human ES cells, and will encourage the use of such ES cells for the isolation of differentiated cells and tissues for transplantation. Specifically, the present invention accomplishes directed differentiation and “de-differentiation” of embryonic and ES cells simultaneously by virtue of genetic modifications that result in ablation of one or more cell lineages. Because the genetic modifications are engineered into the somatic cell nuclear donor before it is used for nuclear transfer, and result in the ablation of entire cell lineages after nuclear transfer, the embryonic and ES cells generated by the methods of the present invention do not have the ability to develop into an embryo. Hence the ES cells of the present invention have no potential for human life.

The de-differentiation methods of the present invention employ genetic modifications that are activated when specific stages of development are reached, i.e., by virtue of cell- or lineage-specific promoters or via stably expressed nucleic acid constructs that have homology to cell- or lineage-specific genes. In particular, the present invention employs RNA interference, a recently identified molecular phenomenon that occurs in a wide variety of cell types, to effect in vivo inhibition of target developmental genes. Thus, there is no need to physically separate cells in vitro to prevent embryo development, and development may be permitted to progress in vivo to allow the isolation of more terminally differentiated cells and tissues. Indeed, because the de-differentiation mechanisms disclosed herein are self-directing, they also facilitate in vivo enrichment of desirable cell types and lineages concomitantly with the cell ablation of other types. Positive selection mechanisms are combined with the negative selection systems to provide for more focused development of differentiated cell types.

The present invention further relates to the use of nuclear transfer embryos, blastocysts, morula, or inner cell mass cells for producing differentiated cells, tissues and organs by culturing in vitro in the presence of appropriate constituents, e.g., grow factors, hormones and other cells without the generation of ES cells and ES cell lines. These embryos may be lineage deficient or normal, and include parthenogenic embryos as well as embryos produced by cross-species nuclear transfer. In a preferred embodiment “helper cells” i.e., cells that induce differentiation into specific cell types, e.g., parenchymal cells, stromal cells or endothelial cells, will be used to induce differentiation of nuclear transfer embryos, blastocysts, morula, inner cell masses, and cells derived from any of the foregoing into differentiated cells and tissues by in vitro co-culture. In a particularly preferred embodiment the nuclear transfer embryos will comprise primate, preferably human embryos.

### BRIEF DESCRIPTION OF THE FIGURES

FIG. 1 shows the formation of differentiated cells (myocardial cells) produced by co-culture of rabbit ICM (parthenogenic) on an endothelial cell monolayer.

FIG. 2 depicts a bioreactor co-culture system used to produce differentiated cells (e.g. myocardial cells) by co-culture of undifferentiated cells (e.g., ICM or ES cells) and helper cells (endothelial cells) according to the invention.

FIG. 3 depicts another bioreactor co-culture system used to produce differentiated cells (e.g., myocardial cells) by co-culture of undifferentiated cells (e.g., ES or ICM cells) and helper cells or other differentiation inducers (e.g., endothelial and stromal cell inducers) according to the invention.

### DETAILED DESCRIPTION OF THE INVENTION

The present invention in part includes methods of making a mammalian nuclear transfer-derived embryos comprising cells that are incapable of differentiating into a particular cell lineage. Because the cells made by the present invention are inherently incapable of developing into a fetus, the nuclear transfer derived embryos made by the present invention and used for therapeutic cloning of tissues never have the potential for human life. In particular, such methods comprise (a) isolating a differentiated mammalian somatic cell to be used as a nuclear transfer donor; (b) genetically engineering said cell to be incapable of differentiating into a particular cell lineage; and (c) effecting nuclear transfer of said differentiated, genetically engineered cell into a suitable recipient cell, thereby forming a mammalian nuclear transfer embryo comprising cells that are incapable of differentiating into a particular cell lineage.

In another embodiment, the invention relates to the production of nuclear transfer embryos, by transplantation of a cell, nucleus, or chromosomes of one cell into a suitable recipient cell, e.g., oocyte or blastomere of the same or different species, to produce a nuclear transfer embryo, and the use of this embryo, or blastocyst, morula, inner cell mass or by parthenogenic or by parthenogenic activation of germ cells (e.g., human oocyte) or cell therefrom to produce desired differentiated cell types by inducing direct differentiation in vitro by culturing in the presence of appropriate growth factors, hormones, minerals, and/or other cells and cell surface factor that promote differentiation. These other cells may be of the same or different species as the nuclear transfer embryo. For example, endothelial, stromal cells, and parenchymal cells may be used. Alternatively, membranes or cell surface molecules can be isolated from such cells or produced by recombinant methods and used to induce differentiation of embryonic cells.

Suitable nuclear transfer donors may be derived from any vertebrate, but preferably will comprise mammalian cells, but in particular will preferably be cells of a human in need of a transplant. Thus, a donor cell may be taken from such a human patient and genetically engineered such that, after using the cell as a nuclear transfer donor, the resulting nuclear transfer unit does not differentiate into one of the three major cell lineages, i.e., endoderm, mesoderm or ectoderm. In the context of therapeutic cloning and the generation of transplantable cells and tissues, the lineage which is precluded from development should of course not be the one which develops into the cells needed for transplantation. The recipient cell may be of the same or different species, preferably an oocyte or a blastomere, that is enucleated prior, simultaneous or after transfer. Suitable donor cells for nuclear transfer include avian, amphibian, reptilian and mammalian cells, nuclei or chromosomes. Such cells may be of any cell cycle, in G₀, G₁, G₂, S or M and of any lineage. Such donor cells include somatic cells and germ cells, e.g., neural, fibroblast, endothelial, cardiac, esophageal, stomach, lymphocytes, primordial, germ cells, cumulus cells, tracheal cells, skin cells, leukocytes, red blood cells, reproduction cells, bladder, urethral, liver, paryenchymal, pancreatic, gall bladder, et al. Such donor cells or DNA therefrom may be haploid, diploid or tetraploid and may be of the same or different species as the recipient cell. As noted, in a preferred embodiment the donor will comprise a human cell or DNA therefrom and the recipient a rabbit or bovine oocyte which is enucleated prior, simultaneous or after transfer.

Thus, the methods of the present invention further comprise permitting the resulting nuclear transfer embryo to differentiate into a desired lineage. Nuclear transfer embryos may be permitted to develop into a morula or blastocyst stage embryo, and such cell lineage deficient embryos or cells derived therefrom, e.g., inner cell mass cells, may be used to isolate the desired differentiated cells. “Desired” differentiated cells will typically be defined in advance according to the needs of a patient for instance, and the genetic modifications made to the somatic cell donor will be designed with such desired cells as an intended goal of concomitant differentiation and de-differentiation. Therefore, differentiation and de-differentiation (or inhibition of the development of a specific cell lineage) occur simultaneously, as precluding the development of a certain lineage by its nature allows the isolation or partial isolation of cells that develop into other lineages. Development into two of the three lineages could also be precluded by the genetic modifications described herein, thereby simultaneously isolating cells that are only capable of developing into one of the three main lineages. Cell lineage deficient nuclear transfer embryos or blastocysts or morulas or inner cell mass cells derived therefrom may be further permitted to differentiate into a desired cell type, as discussed above by the addition of appropriate constituent in vitro.

The methods of the present invention may be used to select any cell type or lineage. Examples of medically relevant cells that could be produced for transplantation therapies include cardiomyocytes (for congestive heart failure and myocardial infarction), hematopoietic stem cells (for the treatment of AIDS patients and patients with diseases or cancers of the blood), endothelial cells (for replacing and repairing blood vessels), pancreatic islet cells (for diabetes), neurons (for Parkinson's, Alzheimer's, stroke patients, etc.), fibroblasts and keratinocytes (for burn patients and wound healing), and chondrocytes or cartilage-forming cells (for replacing joints in rheumatoid arthritis and osteoarthritis patients) (see “The First Derivation of Human Embryonic Stem Cells,” at www.eurekalert.org/releases/ geron_stem_back.html). U.S. application Ser. No. 09/689,743 filed on Oct. 13, 2000, 09/655,815 filed on Sep. 6, 2000 detail the advantages and methods involved in therapeutic cloning using somatic cell nuclear transfer, and are herein incorporated by reference in its entirety.

The differentiated mammalian somatic cell to be used as a nuclear donor may be genetically engineered by physically knocking out a gene required for differentiation into said particular lineage, e.g., using a DNA construct to homologously recombine a deletion or other deleterious modification (insertion, mutation or substitution) into the region of the chromosome where the gene to be controlled is located. Alternatively, the selected donor cell may be genetically engineered by stably transfecting said cell with a suicide gene operably linked to a lineage specific promoter that directs expression of said suicide gene during a particular stage of development. For instance, suicide genes expressed from gene promoters normally expressed in only the endoderm lineage would result in the suicide of all cells that enter the endoderm lineage. Regulatory sequences such as upstream or downstream enhancers, or binding sites for positive regulatory proteins expressed in the suicide-targeted lineage may also be used to direct specific expression of suicide genes.

Possible suicide genes that could be used in this context are known in the art. For instance, thymidine kinase, such as the one from Herpes simplex, phosphorylates GCV, which, in turn, inhibits DNA replication. Another example is cytosine deaminase, which is used in conjunction with 5-fluorocytosine. However, in the case of these suicide genes, precluding development of certain cell lineages requires the administration of GCV or 5-fluorocytosine, whereupon only cells expressing the suicide gene from a lineage specific promoter or other regulatory region will be affected. In this regard, the embryonic cells technically have the capability to achieve life if the drugs are not administered. Moreover, depending on the stage of development of the embryo, the drug has the possibility of affecting non-target cells if either RNA transcripts or products encoded by the transgene travel to neighboring cells, i.e., through gap junctions.

Thus, a more preferable suicide gene would be an apoptosis-inducing gene. Examples of apoptosis-inducing genes include ced genes, myc genes (overexpressed), the bclxs gene, the bax gene, and the bak gene. The apoptosis-inducing gene causes death of transfected cells, i.e., by inducing programmed cell death. For example, the bclxs gene, bax gene, or bak gene can be used to inhibit bcl-2 or bcl-x.sub.L, leading to apoptosis. See U.S. Pat. No. 6,153,184 for disclosure relating to the use of apoptosis genes as suicide genes, which is herein incorporated by reference in its entirety. Where necessary, embryonic cells expressing an apoptosis-inducing gene can be used in combination with an agent that inactivates apoptosis inhibitors such as bcl-z, p35, IAP, NAIP, DAD1 and A20 proteins. This might be desirable, for instance, if one wishes to preclude the development of cells of a particular lineage, but finds that it is necessary to permit the cells targeted for suicide to develop to a certain embryonic stage in order to facilitate the development of desired cells from other cell lineages.

The most preferred method of achieving de-differentiation of specific cell lineages is to stably transfect the donor cell with at least one oligonucleotide operably linked to a promoter or other lineage-specific regulatory sequence, wherein said at least one oligonucleotide encodes an RNA molecule that inhibits or interferes with the expression of at least one gene expressed in the particular lineage that is to be precluded. Said interfering or inhibitory RNA molecule may be an antisense RNA or a ribozyme. When employed, antisense RNAs should be at least about 10-20 nucleotides or greater in length, and be at least about 75% complementary to its target gene or gene transcript such that expression of the homologous gene targeted for de-differentiation is precluded. When employed, ribozymes may be selected from the group consisting of hammerhead ribozymes, axehead ribozymes, newt satellite ribozymes, Tetrahymena ribozymes and RNAse P, and are designed according to methods known in the art based on the sequence of the target gene (for instance, see U.S. Pat. No. 5,741,679, herein incorporated by reference in its entirety).

Preferred RNA molecules of the present invention mediate RNA interference (RNAi) of a target gene or gene transcript. RNAi refers to interference with or destruction of the product of a target gene by introducing a double stranded RNA (dsRNA) that is homologous to the product of a target gene. RNAi was first discovered a couple years ago after one group working with antisense inhibition of a gene in C. elegans found that the control sense RNA also produced a mutant phenotype (Cell 81: 611-20, 1995). It was subsequently discovered that it was the presence of dsRNA in the antisense and control sense RNA preparations that was actually responsible for producing the interfering activity (see J. Travis, “For geneticists, interference becomes an asset,” Science News, Jan. 15, 2000), and that dsRNA is more efficient at silencing the expression of a target gene than a corresponding antisense or sense RNA (see Plasterk and Ketting, 2000, “The silence of the genes,” Current Opinion in Genetics and Dev. 10: 562-67).

It is now known that RNAi is a naturally occurring phenomenon that tightly controls the expression of genes in a wide variety of organisms, including algae, fungi, plants and animals. Researchers have been surprised to find that dsRNA produces specific phenocopies of null mutations in such phylogenetically diverse organisms as Drosophila (Kennerdell and Carthew, 1998, Dev. 95: 1017-26), trypanosomes (Ngo et al., 1998, Proc. Natl. Acad. Sci. USA 95: 14687-92), planaria (Newmark and Sanchez, 1999, Proc. Natl. Acad. Sci. USA 96: 5049-54), and mouse embryos (Svoboda et al., October 2000,Dev. 127(19): 4147-56). It is currently unclear, however, as to whether a single molecular mechanism mediates interference via dsRNA in all organisms, or whether there are different mechanisms that similarly rely on the dsRNA. At least four independent lines of research identify phenomena that relies on the presence of dsRNA—transgene-dependent gene silencing in plants (also termed “co-suppression”), “quelling” in fungi, RNAi in diverse animals and the silencing of transposable elements—with at least one group proposing that all these phenomena are variations of the same molecular mechanism (see Plasterk and Ketting, 2000, supra). On the other hand, another group has found that cosuppression and RNAi have overlapping but distinct genetic requirements (Dernburg et al., 2000, “Transgene-mediated cosuppression in the C. elegans germ line,” Genes Dev. 14(13): 1578-83). To the extent that different molecular variations of gene expression inhibition are mediated by dsRNA, the term RNA interference as used herein should be construed as referring to any or all of these mechanisms.

Although the molecular mechanism of RNAi has not been completely deciphered, current models suggest that the dsRNA must either be replicated or work catalytically since only a few molecules per cell are required to mediate interference (posted at www.macalstr.edu/montgomery/RNAi.html, Dec. 4, 2000).It is proposed that the dsRNA unwinds slightly, allowing the antisense strand to base pair with a short region of the target endogenous message thereby marking it for destruction via degradation. The effect is presumed to be mediated through the transcript of the target gene rather than the gene itself because it only works if the dsRNA is homologous to exon sequences, not intron sequences or promoter sequences (Plasterk and Ketting, 2000, supra). “Marking” mechanisms may involve modifying the target transcript (e.g. by adenosine deaminase or some other mechanism), with a single dsRNA having the capability to mark hundreds of target RNAs for destruction before it is “spent.”

Interestingly, the silencing mechanism RNAi reportedly has the ability to travel or migrate. For instance, in C. elegans, the dsRNA can be taken up in the gut and apparently can migrate from there to the germline where it presumably acts (Plasterk and Ketting, 2000, supra). Whether it is the actual dsRNA that actually “migrates” is unclear, as it has been questioned whether dsRNA is able to cross cell membranes following injection into Drosophila embryos (Clemens et al., 2000, “Use of double-stranded RNA interference in Drosophila cell lines to dissect signal transduction pathways,” Proc. Natl. Acad. Sci. USA 97(12): 6499-6503). Nevertheless, when injected into Drosophila embryos before cellularization (at the syncytial blastoderm stage), the RNAi effect persisted throughout development and could be observed in the adult at low penetrance (Clemens et al., 2000, supra).

The persistence of the interference mediated by dsRNA is ideal for deterring differentiation of targeted cell lineages in the context of the present invention. So long as the dsRNA molecules used to mediate the interference are targeted to the transcript of a gene required for a specific lineage, the effect will be localized to that lineage despite the persistence of the effect, and despite the possible capability of the dsRNA or the molecular mechanism to migrate across cell and tissue barriers. Indeed, the phenomenon has a high degree of specificity for the targeted gene (see Caplen et al., 2000, Gene 252(1-2): 95-105). Moreover, the noted tendency of the effect to “migrate” is ideal for ensuring that the desired block on development is complete, given that the interference will travel to cells of other lineages which might perhaps compensate for the block in development by dividing into cells slated for different lineages.

Prior art reports of the use of RNAi in the context of embryonic development describe the injection of dsRNA into embryos as a means to study embryonic development (e.g., see Kennerdell and Carthew, 1998, “Use of dsRNA-mediated interference to demonstrate that frizzled and frizzled 2 act in the wingless pathway,” Cell 95(7): 1017-26). Although it may be possible to use injection as a means to accomplish the directed de-differentiation of embryonic cell lineages as described in the present invention, this will not resolve the ethical dilemma in that the treated embryos will have the potential for life until the dsRNA is injected. Moreover, the ability of the dsRNA or the effect to travel across cell membranes in a developing embryo is not assured. Although several groups have used injection of dsRNA into embryos, no one has proposed using the technique to direct the development of cells and tissues in the context of therapeutic cloning. Furthermore, no one has proposed using the technique as a means to resolve the ethical dilemma of the short-lived embryo derived in the process of isolating human ES cells.

As different groups have sought ways to overcome the transient effect of injected dsRNA and apply the tool to study more late-acting gene functions in Drosophila, different ways to accomplish heritable transfer of RNAi via stable transfection of various synthetic constructs have recently been proposed. For instance, Kennerdell and Carthew recently reported that hairpin RNA expressed from a transgene was sufficient to mediate RNAi in Drosophila (“Heritable silencing in Drosophila using double-stranded RNA,” Nat. Biotechnol., August 2000, 18(8): 896-8). Similarly, another group reported stable Trypanosoma brucei cell lines expressing inducible dsRNA in the form of stem-loop structures under control of a tetracycline-inducible promoter (Shi et al., July 2000, “Genetic interference in t. brucei by heritable and inducible double-stranded RNA,” RNA 6(7): 1069-76). Another group achieved heat shock-inducible expression of a dsRNA in Drosophila by cloning the target region as a head to head repeat after the hsp70 promoter in a Drosophila P element vector (see Lam and Thummel, August 2000, “Inducible expression of double-stranded RNA directs specific genetic interference in Drosophila,” Curr. Biol. 10(16): 957-63;see also Chuang and Meyerowitz, 2000, “Specific and heritable genetic interference by double-stranded RNA in Arabidopsis thaliana,” Proc. Natl. Acad. Sci. USA 97(9):4985-90). Another group at Johns Hopkins recently reported the inhibition of T. brucei gene expression using an integratable vector with opposing T7 promoters flanking the nucleic acid construct (Wang et al., September 2000, “RNA interference in Trypanosoma brucei,” JBC Papers in Press, Manuscript M008405200). It may also be possible to transfect donor cells with a genetic construct operably linked to a regulatory element specific for an RNA dependent RNA polymerase, whereby the RNA transcript from said construct could be duplicated into dsRNA by said polymerase. An RNA dependent RNA polymerase recognizing the genetic element could be supplied by a separate construct, for instance, one encoding a polymerase cloned from an RNA virus.

Thus, the present invention proposes the use of heritable dsRNA-producing constructs to achieve RNAi in nuclear transfer-derived embryos, and particularly human embryos, in order to facilitate the directed development of human therapeutic tissues for transplantation and ensure that the embryo intermediate has no potential for human life. This may be accomplished using any of the techniques reported in the art, for instance by transfecting a nucleic acid construct encoding a stem-loop or hairpin RNA structure into the genome of the nuclear transfer donor, or by expressing a transfected nucleic acid construct having homology for a target gene from between convergent promoters, or as a head to head or tail to tail duplication from behind a single promoter. Any similar construct may be used so long as it produces a single RNA having the ability to fold back on itself and produce a dsRNA, or so long as it produces two separate RNA transcripts which then anneal to form a dsRNA having homology to a target gene.

Absolute homology is not required for RNAi, with a lower threshold being described at about 85% homology for a dsRNA of about 200 base pairs (Plasterk and Ketting, 200, supra). Therefore, depending on the length of the dsRNA, the nucleic acids of the present invention can vary in the level of homology they contain toward the target gene transcript, i.e., with dsRNAs of 100 to 200 base pairs having at least about 85% homology with the target gene, and longer dsRNAs, i.e., 300 to 100 base pairs, having at least about 75% homology to the target gene. RNA-encoding constructs that express a single RNA transcript designed to anneal to a separately expressed RNA, or single constructs expressing separate transcripts from convergent promoters, are preferably at least about 100 nucleotides in length. RNA-encoding constructs that express a single RNA designed to form a dsRNA via internal folding are preferably at least about 200 nucleotides in length.

The promoter used to express the dsRNA-forming construct may be any type of promoter if the resulting dsRNA is specific for a gene product in the cell lineage targeted for destruction. Alternatively, the promoter may be lineage specific in that it is only expressed in cells of a particular development lineage. This might be advantageous where some overlap in homology is observed with a gene that is expressed in a non-targeted cell lineage. The promoter may also be inducible by externally controlled factors, or by intracellular environmental factors. “Promoter” is intended to encompass any operably linked regulatory sequence, i.e., promoters for gene transcription, or enhancer elements, that contribute to expression of the construct and regulation of that expression.

The methods described herein also include techniques for inducing differentiation and de-differentiation by contacting the nuclear transfer embryos, blastocysts, morulas or inner cell mass cells which may or may not be lineage deficient with one or more growth factors which encourage or deter differentiation, respectively, into a specific cell lineage. The invention also includes the use of the nuclear transfer embryos, blastocysts, morulas, inner cell masses and cells derived therefrom described herein in screening assays and methods for the identification of growth factors which play a role in embryonic development. The cell lineage deficient embryos, blastocysts, etc. of the present invention are particularly suitable for the identification and isolation of such growth factors as they will help reduce the “noise” of such assays by narrowing the scope of cell types induced during differentiation. Such growth factors will help facilitate the isolation of differentiated cells and tissues from non-cell lineage deficient embryonic cells, blastocysts, etc., and are also encompassed in the present invention.

Suitable recipient cells which may be used in the methods of the present invention include vertebrate oocytes, blastomeres or vertebrate ES cells, e.g., mammalian, ES cells such as of human, primate, bovine, porcine, ovine, rabbit, hare, equine, murine, rat, hamster, guinea pig, birds, amphibians and fish. Researchers at Advanced Cell Technology (Worcester, Mass.) have shown that cross-species nuclear transfer of a human nucleus from an adult fibroblast into an enucleated bovine oocyte generates a reprogrammed cell that is capable of several divisions and that human/rabbit oocyte nuclear transfer embryos give rise to blastocyst and ES-like cells. Therefore, it is expected that either cross-species or same species nuclear transfer may be used in the methods of the present invention. Cross-species nuclear transfer technology is described in PCT/US00/05434 and PCT/US00/012631 both of which are herein incorporated by reference.

The method may be used to isolate either an embryonic cell or an embryonic stem cell or a group of such cells. Such cells may further be used to isolate or design therapeutic tissues for transplantation. The embryonic cell or ES cell or group of embryonic cells made by the methods of the present invention are also included, as are any donor cells carrying genetic modifications or dsRNA-producing constructs used for nuclear transfer. In addition, the invention encompasses any further differentiated cells isolated from the directed cell lineages of the present invention, as well as any tissues derived therefrom and methods of transplantation using those tissues.

Any gene expressed specifically in one or two cell lineages and not the other(s) may be used as a target for RNA interference, or for genetic modification according to the invention. For instance, if the particular lineage targeted for de-differentiation is the endoderm lineage, the knockout or RNAi may affect a gene selected from the group consisting of GATA-4, GATA-6, and any other gene specifically expressed in cells of the endoderm lineage. If the particular lineage targeted for de-differentiation is the mesoderm lineage, the knockout or RNAi may affect a gene selected from the group consisting of SRF, MESP-1, HNF-4, beta-1 integrin, MSD, and any other gene specifically expressed in cells of the mesoderm lineage. Alternatively, if the particular lineage targeted for de-differentiation is the ectoderm lineage, the knockout or RNAi may affect a gene selected from the group consisting of RNA helicase A, H beta 58, and any other gene specifically expressed in cells of the ectoderm lineage.

The donor cells of the present invention may be further modified by deleting or modifying at least one harmful or undesirable DNA or by inserting at least one therapeutic or corrective DNA. For instance, where donor cells will be used to replace diseased cells or tissues in a transplant recipient, harmful or undesirable DNA mutations, deletions, etc. may be removed in the donor cell prior to nuclear transfer using well-known recombinant DNA methods. Alternatively, if transplant stability and disease treatment or deterrence would be aided by the insertion of heterologous genes, i.e., genes encoding hormones, enzymes, regulatory proteins, etc., such genes can be inserted into the genome of the donor cell prior to nuclear transfer.

As noted, the invention includes in particular the production of desired differentiated cell types by inducing the differentiation of blastocysts, morula, inner cell masses, or cells derived therefrom into desired cell types in vitro without the production of ES cells. This can be effected in suspension or non-suspension cell culture systems, in the presence or absence of feeder layers.

For example inner cell masses derived from nuclear transfer embryos or from parthenogenic embryos, e.g., by parthenogenic activation of germ cells (oocytes or sperm cells) may be contacted with different combinations of growth factors, hormones, or cells that induce differentiation into specific cell types.

For instance, cells that induce differentiation may be added such as stromal cells derived from developing embryonic and fetal animal tissues of the same or different species. For example, in the case of human inner cell masses produced by same or cross-species nuclear transfer, or by parthenogenesis, stromal cells may be added to an ICM culture, e.g., primate, rabbit, or bovine stromal cells. Such stromal cells may be derived from various tissues, e.g., the brain, eye, pharyngeal pouches, lungs, kidneys, liver, heart, intestine, pancreas, bone, cartilage, skeletal muscle, smooth muscle, ear, esophagus, stomach blood vessels, etc.

In preferred embodiments endothelial cells will be used to induce differentiation of ICMS, blastocysts, morulas, or cells derived therefrom, preferably human blastocysts, morulas, inner cell masses, or cells derived therefrom.

For instance, fetal or embryonic liver endothelial cells may be used to induce differentiation of undifferentiated cells into hematopoietic stem cells, preferably repopulating hematopoietic stem cells. The resultant hematopoietic stem cells may be used to treat patients wherein such cells are depleted, e.g., patients undergoing chemotherapy, radiotherapy or which have a disease or genetic defect that results in aberrant numbers of or abnormal hematopoietic stem cells. For instance, such cells may be transplanted into patients with immunodeficiencies that deplete such cells.

The production of such hematopoietic of such hematopoietic stem cells may be effected in culture, e.g., a endothelial monolayer culture unto which ES cells, ICM, or cells derived from a blastocyst or morula are placed, and co-cultured. This may be effected by placing such cells on or proximate to the endothelial monolayer on a tissue culture dish, allowing for cell-cell communication. As noted, the endothelial or other “helper cell”, i.e., cell that promotes differentiation, may be of the same or differentiation species as the ICM, blastocyst, or morula cells. In some instances, the cell culture may comprise several different types of helper cells, e.g., to promote tissue or organ development in vitro.

In another embodiment, endothelial cells may be used to induce differentiation of ICMs, ES cells, blastocyst or morula cells into myocardial cells, e.g., by co-culture with endothelial cells derived from fetal heart, e.g., non-human primate, rabbit, murine, rat, bovine, hamster, ovine, porcine, etc. In a preferred embodiment, the co-culture will comprise endothelial cells derived from rabbit fetal heart tissue, by co-culture of such cells with human ES, ICM, blastocyst or morula cells, produced by nuclear transfer or parthenogenic activation of human germ cells (oocytes). The latter may be preferred as such cells are incapable of giving rise to viable offspring, but still differentiation into all through germ layers.

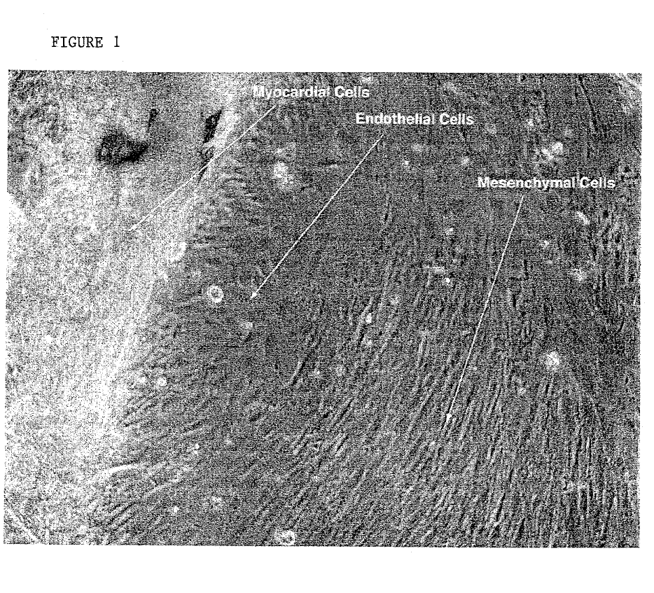

In particular, it has been shown by the inventors that beating myocardial cells (see FIG. 1) may be obtained by culturing ICM produced by parthenogenesis (activation of rabbit oocyte) or an endothelial cell monolayer.

Endothelial cells, or other cells that induce myocardial differentiation can be isolated from spontaneous mutates of myocardial development from such cultures. Isolation may be effected by labeling with DII-labeled LDC that is specifically taken up by vascular endothelial cells.

The cells are then removed from the culture, and flow-sorted and the DII labeled cells are replaced as a relatively pure population of endothelial cells. Endothelial cells that induce differentiation are propagated in vitro, cryopreserved and used in screening assays to induce myocardial differentiation, or to produce myocardial cells for research or therapy.

The invention further contemplates the production of artificial organs and tissues in vitro by use of three-dimensional bioreactor. For example, endothelial or other cells that induce differentiation into specific cell types, e.g., myocardial cells, may be added to three-dimensional bioreactors containing ICM, blastocyst, morula, or ES cells. In one embodiment endothelial cells that induce myocardial differentiation are trypsinized, and permitted to attach to polymer tubes or vessels that promote vascularization and the development of blood vessels. These tubes also allow media to perform and support endothelial attachment and cell viability. In particular, these artificial vessels will be perfused with tissue culture media containing factors that promote the growth of helper cells, e.g., endothelial and which promote differentiation into a desired cell type, e.g., a desired cell type, e.g., myocardial cells. For example, in the case of myocardial cells, the media may comprise brain-derived growth factor (BDNF), or vascular endothelial growth factor-A (VEGF-A), preferably isoform 165.

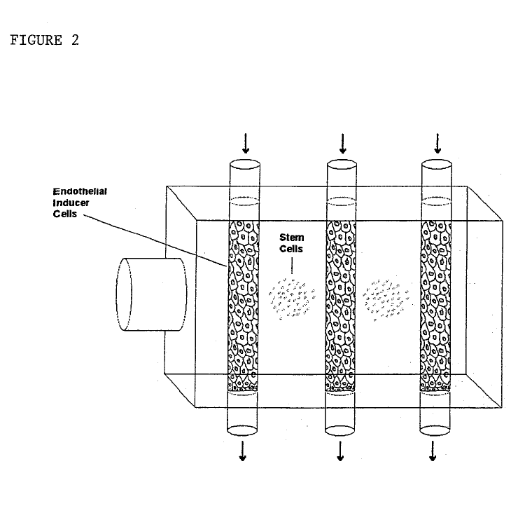

This approach will work with different endothelial cell types to give rise to different types of tissues. Such endothelial cells may be embryonic, fetal or adult and include those already identified. The invention further embraces the tissues generated using these three-dimensional bioreactors, which optionally may be transgenic. Such a three-dimensional culture system is depicted schematically in FIG. 2.

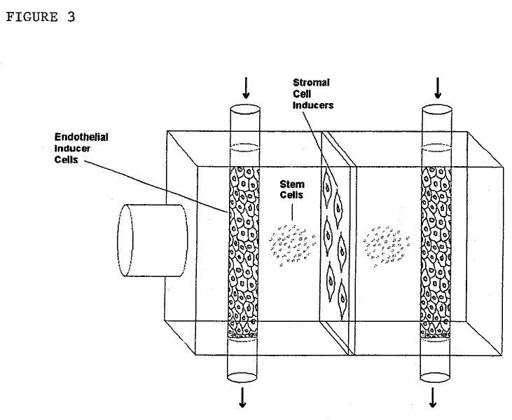

The invention further embraces the combination of endothelial cells that induce differentiation with stromal (e.g., fibroblast) cell inducers. An example of this embodiment of the invention is shown schematically in FIG. 3.

Such a system may be used with many different endothelial and stromal cell types in order to generate desired cells and three-dimensional tissues. The endothelial and stromal cells can be of the same tissue of origin and may be derived from different tissues, and may be of the same or different species as the ES, ICM, morula, or blastocyst cells that are co-cultured therewith. Such cells may be genetically modified and can be of embryonic, fetal or adult origin. Potential types of endothelial and stromal cells include by way of example kidney, liver, brain, heart, intestine, pancreas, stomach, eye, bone, skin, lung, etc.

As depicted in FIG. 3, a co-culture according to the invention will comprise endothelial, stromal cell inducers on a membrane and undifferentiated cells, e.g., ICM, blastocyst, morula, or ES cells, preferably of human origin. In an especially preferred embodiment such cells will be obtained by parthenogenic activation of human oocytes or by cross-species nuclear transfer, e.g., by transplantation of a human cell, nuclear or chromosomes into a rabbit oocyte, which is enucleated before, simultaneous or after transfer. Of course, the bioreactors in FIGS. 2 and 3 are intended to be exemplary as such bioreactors can take various forms in order to grow tissues in two or three dimensions. Bioreactors which are useful for producing tissues exhibiting desired morphology and tissue architecture are known in the art.

Another embodiment of the invention includes the marking of human undifferentiated cells with marker genes that are expressed in differentiated progeny of such cells. Thereby genes which are turned on upon differentiation may be identified. For example, such cells may be produced by transfecting human donor cells with selectable marker genes, e.g., green fluorescent protein (GFP) DNA sequences. Genes that “light up” on cell differentiation will comprise those that are involved with and/or promote differentiation.

As noted, the co-culture aspect of the invention includes the addition of cell surface molecules that facilitate differentiation of undifferentiated cells, e.g., which may be added as isolated proteins, DNA or RNAs, or as membrane extracts, e.g., membrane blebs derived from helper cells, e.g., endothelial stromal, and parenchymal cells.

The invention further embraces the use of helper cells (cells that induce differentiation) that are capable of cell division or which are arrested in their growth by various means, e.g., radiation, DNA damaging agents, viral invention, and others.

In yet another embodiment the bioreactors and subject co-culture method may be used to provide the actual vasculature, i.e., perfusion of resulting tissue. Thereby, the subject bioreactor co-culture system may be used to produce artificial and vascularized organs, e.g., artificial pancreas for treatment of diabetes.

As discussed the bioreactor can take various forms, e.g., coated cylinders, tissue culture plates and dishes, comprising undifferentiated cells, helper cells and appropriate media to induce cell differentiation, e.g., of ICMS, blastocyst or morula cells.

Further derivations of the above-described invention may be envisioned by the reader, and are included within the scope of the disclosed invention.

What is claimed:

1. A method of making a mammalian nuclear transfer embryo that is comprised of cells that are incapable of differentiating into a particular cell lineage, comprising:
(a) isolating a differentiated mammalian cell to be used as a nuclear transfer donor;
(b) genetically engineering said cell to be incapable of differentiating into a particular cell lineage;
(c) effecting nuclear transfer of said differentiated, genetically engineered cell, nucleus or chromosomal DNA thereof into a suitable recipient cell;
thereby forming a nuclear transfer embryo comprised of cells that are incapable of differentiating into a particular cell lineage.

2. The method of claim 1, wherein said nuclear transfer embryo is permitted to develop into a blastocyst or morula.

3. The method of claim 2, wherein said blastocyst, morula or cells derived therefrom are permitted to differentiate.

4. The method of claim 1, wherein said differentiated mammalian cell is a human cell.

5. The method of claim 1, wherein said particular cell lineage into which said nuclear transfer embryo is incapable of differentiating is selected from the group consisting of endoderm, mesoderm and ectoderm lineages.

6. The method of claim 5, wherein said particular lineage is more specifically selected from the group consisting of cardiomyocytes, hematopoietic stem cells, endothelial cells, pancreatic islet cells, neurons, fibroblasts and keratinocytes, and chondrocytes.

7. The method of claim 5, wherein said differentiated mammalian cell is genetically engineered by knocking out a gene required for differentiation into said particular lineage.

8. The method of claim 1, wherein said differentiated mammalian cell is genetically engineered by stably transfecting said cell with a suicide gene operably linked to a lineage specific promoter expressed during said particular stage of development.

9. The method of claim 5, wherein said differentiated mammalian cell is genetically engineered by stably transfecting said cell with at least one oligonucleotide operably linked to a promoter, wherein said at least one oligonucleotide encodes an RNA molecule that inhibits or interferes with the expression of at least one gene expressed in said particular lineage.

10. The method of claim 9, wherein said interfering or inhibitory RNA molecule is selected from the group consisting of antisense RNAs, ribozymes and RNA molecules that mediate RNA interference (RNAi) of a target gene or gene transcript.

11. The method of claim 10, wherein said RNA molecule is an antisense RNA that is about 10 to 20 nucleotides or greater in length.

12. The method of claim 10, wherein said RNA molecule is an antisense RNA, and is at least about 75% complementary to its target gene or gene transcript.

13. The method of claim 10, wherein said RNA molecule is a ribozyme selected from the group consisting of hammerhead ribozymes, axehead ribozymes, newt satellite ribozymes, Tetrahymena ribozymes and Rnase P.

14. The method of claim 10, wherein said RNA molecule mediates RNAi of a target gene, and is at least about 100 nucleotides in length.

15. The method of claim 14, wherein said differentiated mammalian cell is genetically engineered with a second RNA molecule that mediates RNAi and is also expressed from an oligonucleotide operably linked to a promoter, wherein said second RNA molecule forms a double stranded RNA with said first RNA molecule following expression, thereby effecting RNAi against the target gene or gene transcript.

16. The method of claim 15, wherein said first and second RNA molecules are expressed from the same gene construct operably linked on either end to convergent promoters such that each promoter directs transcription of the opposite strand of the gene.

17. The method of claim 10, wherein said RNA molecule mediates RNAi of a target gene, and forms a stem-loop or hairpin structure.

18. The method of claim 17, wherein said RNA molecule is at least-about 200 nucleotides in length.

19. The method of claim 9, wherein said promoter is lineage specific in that it is only expressed during said particular development lineage.

20. The method of claim 19, wherein said promoter is inducible.

21. The method of claim 1, wherein said suitable recipient cell is a mammalian oocyte or ES cell selected from the group consisting of human, primate, bovine, porcine, sheep, goat, rat, mouse, hamster, guinea pig, horse, birds, amphibians and fish.

22. The method of claim 1, wherein the cells derived from said blastocyst or morula are inner cell mass cells.

23. The cell lineage deficient nuclear transfer embryo made by the method of claim 1.

24. Cell lineage deficient embryonic stem cells derived from the inner cell mass cells of claim 22.

25. The method of claim 7, wherein said particular lineage is the endoderm lineage, and said knockout affects a gene selected from the group consisting of GATA-4 and GATA-6.

26. The method of claim 7, wherein said particular lineage is the mesoderm lineage, and said knockout affects a gene selected from the group consisting of SRF, MESP-1, HNF-4, beta-1 integrin and MSD.

27. The method of claim 7, wherein said particular lineage is the ectoderm lineage, and said knockout affects a gene selected from the group consisting of RNA helicase A and H beta 58.

28. The method of claim 9, wherein said particular lineage is the endoderm lineage, and said at least one gene is selected from the group consisting of GATA-4 and GATA-6.

29. The method of claim 9, wherein said particular lineage is the mesoderm lineage, and said at least one gene is selected from the group consisting of SRF, MESP-1, HNF-4, beta-1 integrin and MSD.

30. The method of claim 9, wherein said particular lineage is the ectoderm lineage, and said at least one gene is selected from the group consisting of RNA helicase A and H beta 58.

31. A human somatic or embryonic cell comprising a heterologous DNA construct or constructs, wherein expression of said heterologous DNA construct or constructs results in a double-stranded RNA molecule that mediates RNA interference (RNAi) of a target gene expressed during embryonic development.

32. The human somatic or embryonic cell of claim 31, wherein said target gene is expressed during a particular cell lineage selected from the group consisting of endoderm, mesoderm and ectoderm.

33. The human somatic or embryonic cell of claim 31, wherein said heterologous DNA construct or constructs are expressed from a lineage specific promoter or promoters.

34. The human somatic or embryonic cell of claim 31, wherein said heterologous DNA construct and constructs are expressed from an inducible promoter or promoters.

35. The human somatic or embryonic cell of claim 31, wherein said double stranded RNA molecule results from hairpin annealing of a single RNA transcript.

36. The human somatic or embryonic cell of claim 31, wherein said double stranded RNA molecule results from annealing of two separate RNA transcripts.

37. A method of making a nuclear transfer embryo comprising cells that are incapable of differentiating into a particular cell lineage, comprising:
(a) isolating a differentiated mammalian cell to be used as a nuclear transfer donor;
(b) stably transfecting into said cell one or more nucleic acid constructs that result in or mediate RNA interference (RNAi) of a target gene expressed in said particular cell lineage;
(c) effecting nuclear transfer of said differentiated, genetically engineered cell, nucleus or chromosomal DNA therefrom into a suitable recipient cell;
thereby forming a nuclear transfer embryo comprising cells that are incapable of differentiating into said particular cell lineage.

38. The method of claim 37, wherein said double stranded RNA molecule is formed via hairpin or stem-loop formation from a single RNA transcript.

39. The method of claim 37, wherein said double stranded RNA molecule is formed by the annealing of separate RNA transcripts.

40. The method of claim 39, wherein said separate RNA transcripts are expressed from the same double stranded DNA construct that is flanked by convergent promoters.

41. The method of claim 37, wherein said differentiated mammalian cell is a human cell.

42. The method of claim 41, wherein said suitable recipient cell is a mammalian oocyte or ES cell selected from the group consisting of human, primate, bovine, porcine, sheep, goat, rat, mouse, hamster, guinea pig, horse, birds, amphibians and fish.

43. The method of claim 37, wherein said nuclear transfer embryo is incapable of differentiating into a cell lineage selected from the group consisting of endoderm, mesoderm and ectoderm.

44. The transfected differentiated mammalian cell formed in step (b) of claim 37.

45. The cell lineage deficient nuclear transfer embryo made by the method of claim 37.

46. The human cell lineage deficient nuclear transfer embryo made by the method of claim 41.

47. The method of claim 37 further comprising permitting said nuclear transfer embryo to develop into a morula or blastocyst.

48. The method of claim 47, wherein said blastocyst, morula or cells derived therefrom are permitted to differentiate.

49. The method of claim 48, wherein the cells derived from said morula or blastocyst are inner cell mass cells.

50. Cell lineage deficient embryonic stem cells derived from the inner cell mass cells of claim 49.

51. Differentiated cells made by the method of claim 3.

52. A tissue engineered using the differentiated mammalian cells of claim 51.

53. Differentiated cells made by the method of claim 48.

54. A tissue engineered using the differentiated human cells of claim 53.

55. The method of claim 1, further comprising a step between steps (b) and (c) wherein said differentiated mammalian donor cell is further genetically engineered by deleting or modifying at least one harmful or undesirable DNA or by inserting at least one therapeutic or corrective DNA.

56. The method of claim 37, further comprising a step between steps (b) and (c) wherein said differentiated mammalian donor cell is further genetically engineered by deleting or modifying at least one harmful or undesirable DNA or by inserting at least one therapeutic or corrective DNA.

57. The method of claim 41, further comprising a step between steps (b) and (c) wherein said differentiated human donor cell is further genetically engineered by deleting or modifying at least one harmful or undesirable DNA or by inserting at least one therapeutic or corrective DNA.

58. The genetically modified human nuclear transfer embryo isolated by the method of claim 56.

59. The genetically modified nuclear transfer embryo isolated by the method of claim 57.

60. A method of isolating genetically modified differentiated human cells of a desired lineage comprising growing the nuclear transfer embryo of claim 58 in such a manner as to permit differentiation into a desired lineage.

61. The genetically modified differentiated human cells of a desired lineage isolated by the method of claim 60.

62. A tissue engineered using the genetically modified differentiated human cells of claim 61.

63. A method of therapy using the differentiated mammalian cells of a desired lineage of claim 51.

64. A method of therapy using the differentiated mammalian cells of a desired lineage of claim 53.

65. A method of therapy using the genetically modified differentiated human cells of a desired lineage of claim 61.

66. A method of transplantation using the engineered mammalian tissue of claim 52.

67. A method of transplantation using the engineered human tissue of claim 54.

68. A method of transplantation using the genetically modified engineered human tissue of claim 62.

69. The method of claim 1, wherein said differentiated mammalian cell nuclear transfer donor is either a somatic cell or an embryonic cell.

70. The method of claim 37, wherein said differentiated mammalian cell nuclear transfer donor is either a somatic cell or an embryonic cell.

71. The method of claim 37, wherein said one or more nucleic acid constructs that result in or mediate RNAi include DNA constructs that upon expression result in the formation of a double stranded molecule, and single stranded or double stranded RNA molecules.

72. The method of claim 71, wherein said DNA constructs either integrate into the chromosome or are expressed episomally.
